(19) United States
(12) Patent Application Publication
(10) Pub. No.: US 2023/0049130 A1
(43) Pub. Date: Feb. 16, 2023

(54) DEUCRAVACITINIB CRYSTAL FORM, PREPARATION METHOD THEREFOR AND USE THEREOF

(71) Applicant: CRYSTAL PHARMACEUTICAL (SUZHOU) CO., LTD., Jiangsu (CN)

(72) Inventors: Minhua CHEN, Jiangsu (CN); Hongyan ZHU, Jiangsu (CN)

(21) Appl. No.: 17/787,992

(22) PCT Filed: Dec. 28, 2020
(86) PCT No.: PCT/CN2020/139815

§ 371 Date: Jun. 22, 2022

(30) Foreign Application Priority Data
Jan. 19, 2020 (CN) 202010061688.X

(51) Int. Cl. C07D 403/12

(52) U.S. Cl. CPC C07D 403/12; C07B 2200/13

(57) ABSTRACT

Disclosed are a crystal form of a compound (I) and a preparation method therefor, a pharmaceutical composition containing the crystal form, and the use of the crystal form in the preparation of a TYK2 inhibitor drug and a drug for treating psoriasis, systemic lupus erythematosus, and Crohn’s disease. The crystal form of compound I has one or more improved properties compared with the prior art and is of great value to the future optimization and development of the drug.

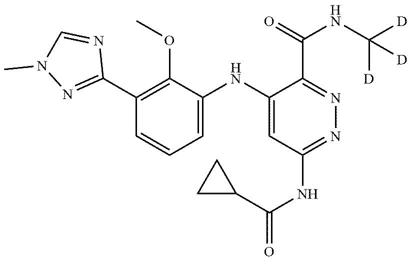 [SMILES: [2H]C([2H])([2H])NC(=O)c1nnc(NC(=O)C2CC2)cc1Nc1cccc(-c2ncn(C)n2)c1OC]

### TECHNICAL FIELD

The present disclosure pertains to the field of chemical crystallography, particularly relates to a crystalline form of BMS-986165, preparation method and uses thereof.

### BACKGROUND

Tyrosine kinase 2 (TYK2) is an intracellular signaling kinase that mediates signaling of interleukin-23 (IL-23), interleukin-12 (IL-12) and Type I interferon (IFN), which are cytokines involved in inflammatory and immune responses.

BMS-986165 is the first and only novel, oral, selective TYK2 inhibitor which is used for the treatment of multiple immune-mediated diseases such as psoriasis, psoriatic arthritis, lupus and inflammatory bowel disease. The phase III clinical study results announced in November 2020 reveal that BMS-986165 shows positive clinical effects in the treatment of moderate to severe plaque psoriasis. In addition, BMS-986165 also shows good therapeutic effects in treatment of systemic lupus erythematosus and Crohn’s disease.

The chemical name of BMS-986165 is 6-(cyclopropanecarboxamido)-4-((2-methoxy-3-(1-methyl-1H-1,2,4-triazol-3-yl) phenyl) amino)-N-(methyl-d3) pyridazine-3-carboxamide (hereinafter referred to as “Compound I”), and the structure is shown as follows:

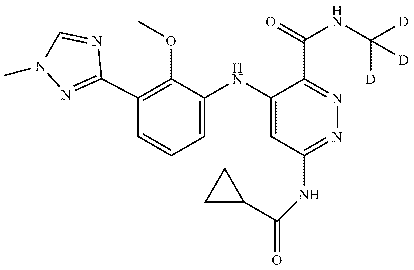 [SMILES: [2H]C([2H])([2H])NC(=O)c1nnc(NC(=O)C2CC2)cc1Nc1cccc(-c2ncn(C)n2)c1OC]

A crystalline form is a solid material whose constituents are arranged in a highly ordered microscopic structure, forming a crystal lattice that extends in all directions. Polymorphism refers to the phenomenon that a compound exists in two or more than two crystalline forms. Different crystalline forms have different physicochemical properties and can affect drug’s in vivo dissolution and absorption, which further affect drug’s clinical efficacy and safety to some extent. In particular, for poorly soluble drugs, the effects of crystalline forms will be greater. Therefore, drug polymorphism is an important part of drug research and drug quality control.

WO2018183656A1 disclosed crystalline form A of Compound I (hereinafter referred to as Form A) and process for preparation. Form A disclosed in WO2018183656A1 is the only known crystalline form of Compound I free form. The inventors of the present disclosure repeated the preparation method disclosed in WO2018183656A1 to obtain Form A and conducted characterizations. The results show that Form A has poor solubility and low density. The poor solubility may affect the bioavailability of the drugs. Therefore, it is still necessary to develop a crystalline form of Compound I, which has high solubility and good stability, for the development of drugs containing Compound I.

The inventors of the present disclosure put a lot of creative work, and then surprisingly discovered crystalline form CSIII of Compound I provided by the present disclosure, which has advantages in physiochemical properties, formulation processability, bioavailability, etc. For example, crystalline form CSIII of Compound I has advantages in at least one aspect of melting point, solubility, hygroscopicity, purification ability, stability, adhesiveness, compressibility, flowability, in vitro and in vivo dissolution, and bioavailability, etc. In particular, crystalline form CSIII of Compound I has high solubility, good physicochemical stability, good mechanical stability, high density, which solves the problems existing in prior arts and is of great significance for the development of drugs containing Compound I.

### SUMMARY

The main objective of the present disclosure is to provide a novel crystalline form of Compound I, preparation method and use thereof.

According to the objective of the present disclosure, crystalline form CSIII of Compound I is provided (hereinafter referred to as Form CSIII).

In one aspect provided herein, the X-ray powder diffraction pattern of Form CSIII comprises characteristic peaks at 2theta values of 6.4°±0.2°, 11.3°±0.2°and 23.2°±0.2° using CuKα radiation.

Furthermore, the X-ray powder diffraction pattern of Form CSIII comprises one or two or three characteristic peaks at 2theta values of 20.6°±0.2°, 25.9°±0.2° and 27.8°±0.2° using CuKα radiation; preferably, the X-ray powder diffraction pattern of Form CSIII comprises three characteristic peaks at 2theta values of 20.6°±0.2°, 25.9°±0.2° and 27.8°±0.2° using CuKα radiation.

In another aspect provided herein, the X-ray powder diffraction pattern of Form CSIII comprises three or four or five or six or seven or eight or nine characteristic peaks at 2theta values of 6.4°±0.2°, 11.3°±0.2°, 23.2°±0.2°, 10.1°±0.2°, 12.7°±0.2°, 19.3°±0.2°, 20.6°±0.2°, 25.9°±0.2° and 27.8°±0.2° using CuKα radiation.

Without any limitation being implied, the X-ray powder diffraction pattern of Form CSIII is substantially as depicted in FIG. 1.

Without any limitation being implied, the Differential Scanning Calorimetry (DSC) curve of Form CSIII is substantially as depicted in FIG. 2, which shows an endothermic peak at around 257° C. (onset temperature). This peak is the melting endothermic peak.

Without any limitation being implied, the Thermo Gravimetric Analysis (TGA) curve of Form CSIII is substantially as depicted in FIG. 3, which shows about 0.4% weight loss when heated to 200° C.

Without any limitation being implied, Form CSIII is an anhydrate.

According to the objective of the present disclosure, a process for preparing Form CSIII is also provided. The process comprises: dissolving Compound I solid in an amide, evaporating to obtain Form CSIII.

Furthermore, said amide is preferably N, N-dimethylformamide or a solvent mixture of N, N-dimethylformamide and N, N-dimethylacetamide, said evaporating temperature is preferably 40° C.-80° C., further preferably 50° C.

Form CSIII of the present disclosure has the following advantages:

(1) Compared with prior arts, Form CSIII of the present disclosure has a higher solubility. In particular, the solubility of Form CSIII equilibrated in FaSSIF medium for 1 hour and 4 hours are 3.6 and 4.1 times that of Form A, respectively.

Compound I is a poorly water-soluble drug. Higher solubility is beneficial to improve drug’s in vivo absorption and bioavailability, thus improving drug efficacy. In addition, drug dose reduction without affecting efficacy is possible due to higher solubility, thereby reducing the drug’s side effects and improving drug safety.

(2) Form CSIII drug substance of the present disclosure has good stability. Crystalline state of Form CSIII drug substance doesn’t change for at least 3 months when stored under 25° C./60%RH with open or sealed condition. The chemical purity remains substantially unchanged during storage. These results show that Form CSIII drug substance has good stability under long-term storage condition, which is beneficial to the drug storage.

Meanwhile, crystalline state of Form CSIII drug substance doesn’t change for at least 3 months when stored under 40° C./75%RH with open and sealed condition. The chemical purity remains substantially unchanged during storage. Crystalline state of Form CSIII drug substance doesn’t change for at least one month when stored under 60° C./75% RH with open and sealed condition. The chemical purity changes only by 0.07% with open condition, which remains substantially unchanged during storage. These results show that Form CSIII drug substance has good stability under accelerated and stress conditions. Drug substance will go through high temperature and high humidity conditions caused by different season, regional climate and environment during storage, transportation, and manufacturing processes. Therefore, good stability under accelerated and stress conditions is of great importance to the drug development. Form CSIII drug substance has good stability under stress condition, which is beneficial to avoid the influence on drug quality when not stored under condition recommended in label.

Form CSIII of the present disclosure has good physical stability. The crystalline state of From CSIII remains unchanged after being stored under a humidity cycle of 0%-95%-0% RH.

Meanwhile, Form CSIII has good mechanical stability. Crystalline state of Form CSIII drug substance doesn’t change after grinding. Form CSIII drug substance has good physical stability. Grinding and pulverization are often required in the drug manufacturing process. Good physical stability of the drug substance can reduce the risk of crystallinity decrease and crystal transformation during the drug production process. Form CSIII drug substance has good physical stability under different pressures, which is beneficial to keep crystalline form stable during tableting process.

Crystal transformation can lead to changes in the absorption of the drug, affect bioavailability, and even cause toxicity and side effects. Good chemical stability of drug substance ensure that no impurities are generated during production and storage. Form CSIII has good physical stability, ensuring consistent and controllable quality of the drug substance and drug product, minimizing quality change, bioavailability change and toxicity due to crystal transformation or impurity generation.

(3) Compared with prior arts, Form CSIII of the present disclosure has higher density. Test results indicate that the bulk density and tapped density of Form CSIII are remarkably higher than that of Form A. Higher density of Form CSIII is beneficial to large scale production. Higher density of Form CSIII can also reduce dust, reduce occupational hazard and ensure production safety.

According to the objective of the present disclosure, a pharmaceutical composition is provided, said pharmaceutical composition comprises a therapeutically effective amount of Form CSIII and pharmaceutically acceptable excipients.

Furthermore, Form CSIII can be used for preparing TYK2 inhibitor drugs.

Furthermore, Form CSIII can be used for preparing drugs treating psoriasis, systemic lupus erythematosus, and Crohn’s disease.

Said “drying” is accomplished at room temperature or a higher temperature. The drying temperature is from room temperature to about 100° C., or to 60° C., or to 50° C. The drying time can be 0.5 to 48 hours, or overnight. Drying is accomplished in a fume hood, forced air convection oven or vacuum oven.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “characteristic peak” refers to a representative diffraction peak used to distinguish crystals, which usually can have a deviation of ±0.2° using CuKα radiation.

In the present disclosure, “crystal” or “crystalline form” refers to the crystal or the crystalline form being identified by the X-ray diffraction pattern shown herein. Those skilled in the art are able to understand that the experimental errors depend on the instrument conditions, the sample preparation and the purity of samples. The relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSIII of Compound I of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight o, time, temperature, and the like, is meant to encompass variations of ± 10%, ± 5%, ± 1%, ± 0.5%, or even ± 0.1% of the specified amount.

### BRIEF DESCRIPTION OF THE DRAWINGS

FIG. 1 shows an XRPD pattern of Form CSIII in Example 1.

FIG. 2 shows a DSC curve of Form CSIII in Example 1.

FIG. 3 shows a TGA curve of Form CSIII in Example 2.

FIG. 4 shows an XRPD pattern overlay of Form CSIII stored under different conditions (from top to bottom: initial, 25° C./60%RH for 3 months (sealed), 25° C./60%RH for 3 months(open), 40° C./75%RH for 3 months (sealed), 40° C./75%RH for 3 months (open), 60° C./75%RH for one month (sealed), 60° C./75%RH for one month (open)).

FIG. 5 shows an XRPD pattern overlay of Form CSIII before and after tableting under different pressures (from top to bottom: initial, tableting under 5 kN, tableting under 10 kN, tableting under 20 kN).

FIG. 6 shows an XRPD pattern overlay of Form CSIII before and after grinding (top: after grinding, bottom: before grinding).

FIG. 7 shows an XRPD pattern overlay of Form CSIII before and after DVS test (top: before DVS, bottom: after DVS).

### DETAILED DESCRIPTION

The present disclosure is further illustrated by the following examples which describe the preparation and use of the crystalline forms of the present disclosure in detail. It is obvious to those skilled in the art that many changes in the materials and methods can be accomplished without departing from the scope of the present disclosure.

The abbreviations used in the present disclosure are explained as follows:

XRPD: X-ray Powder Diffraction

DSC: Differential Scanning Calorimetry

TGA: Thermo Gravimetric Analysis

1H NMR: Proton Nuclear Magnetic Resonance

HPLC: High Performance Liquid Chromatography

DVS: Dynamic Vapor Sorption

Instruments and methods used for data collection:

X-ray powder diffraction patterns in the present disclosure examples 1 and 7-8 were acquired by a Bruker D2 PHASER X-ray powder diffractometer. The parameters of the X-ray powder diffraction method of the present disclosure are as follows:
X-ray source: Cu, Kα
Kα1 (Å): 1.54060; Kα2 (Å): 1.54439
Kα2/Kα1 intensity ratio: 0.50
Voltage: 30 (kV)
Current: 10 (mA)
Scan range: from 3.0 degree to 40.0 degree

X-ray powder diffraction patterns in the present disclosure examples 4-6 were acquired by a Bruker D8 DISCOVER X-ray powder diffractometer. The parameters of the X-ray powder diffraction method of the present disclosure are as follows:
X-ray source: Cu, Kα
Ka1 (Å): 1.54060; Kα2 (Å): 1.54439
Kα2/Kα1 intensity ratio: 0.50
Voltage: 40 (kV)
Current: 40 (mA)
Scan range: from 4.0 degree to 40.0 degree

DSC data in the present disclosure were acquired by a TA Q2000. The parameters of the DSC method of the present disclosure are as follows:
Heating rate: 10° C./min
Purge gas: N₂

TGA data in the present disclosure were acquired by a TA Q500. The parameters of the TGA method of the present disclosure are as follows:
Heating rate: 10° C./ min
Purge gas: N₂

Dynamic Vapor Sorption (DVS) is measured via an SMS (Surface Measurement Systems Ltd.) intrinsic DVS instrument. Its control software is DVS- Intrinsic control software, and its analysis software is DVS-Intrinsic Analysis software. Typical Parameters for DVS test are as follows:
Temperature: 25° C.
Gas and flow rate: N₂, 200 mL/min
RH range: 0% RH to 95% RH

Proton nuclear magnetic resonance spectrum data (¹H NMR) were collected from a Bruker Avance II DMX 400 M Hz NMR spectrometer. 1-5 mg of sample was weighed and dissolved in 0.5 mL of deuterated chloroform to obtain a solution with a concentration of 2-10 mg/mL.

The parameters for kinetic solubility and related substance detection in the present disclosure are shown in Table 1.

Table 1

| | | |
|---|---|---|
| HPLC | Waters ACQUITY H-class UPLC with PDA detector | |
| Column | Waters ACQUITY UPLC BEH C18, 2.1×50 mm, 1.7 µm | |
| Mobile Phase | A: 0.1 % Trifluoroacetic acid (TFA) in H₂O (v/v) | |
| | B: 0.1 % TFA in Acetonitrile (v/v) | |
| Gradient | Time (min) | % B |
| | 0.0 | 10 |
| | 0.5 | 10 |
| | 7.0 | 80 |
| | 8.0 | 80 |
| | 8.1 | 10 |
| | 10.0 | 10 |
| Running time | 10.0 min | |
| Stop time | 0 min | |
| Speed | 0.5 mL/min | |
| Injection Volume | 1 µL | |
| Detection wavelength | UV at 240 nm | |
| Column Temperature | 40° C. | |
| Sample Temperature | Room Temperature | |
| Diluent | Acetonitrile: H₂O = 50:50 (v/v) | |

Unless otherwise specified, the following examples were conducted at room temperature. Said “room temperature” is not a specific temperature, but a temperature range of 10-30° C.

According to the present disclosure, Compound I used as a raw material includes but not limited to solid (crystalline or amorphous), oil, liquid and solution. Preferably, Compound I used as the raw material is a solid.

Compound I used in the following examples can be prepared by known methods in the prior arts, for example, the method disclosed in WO2018183656A1.

### EXAMPLES

### Example 1 Preparation of Form CSIII

10.4 mg of Compound I was weighed into a glass vial, and then 0.5 mL of N, N-dimethylformamide was added to form a clear solution. The solution was filtered and the filtrate was evaporated at 50° C. to obtain a crystalline solid.

The crystalline solid was confirmed to be Form CSIII of the present disclosure. The XRPD pattern of Form CSIII is shown in FIG. 1, and the XRPD data are listed in Table 2.

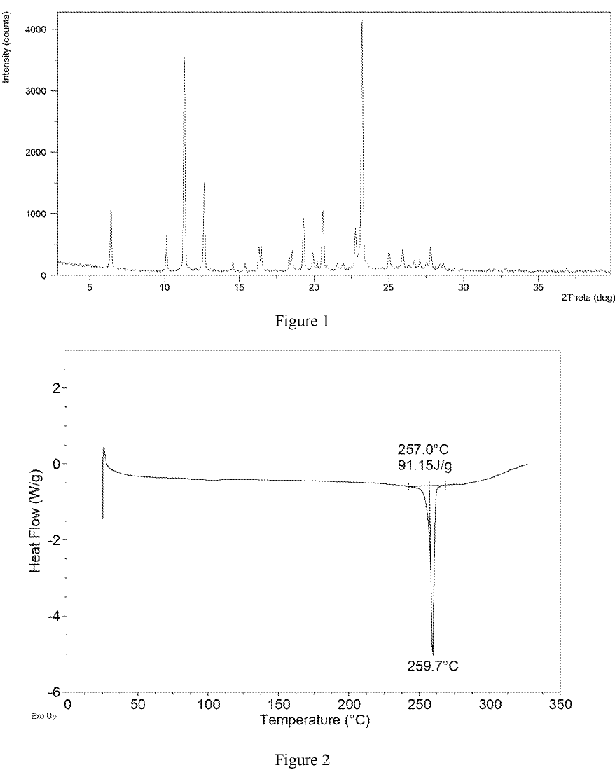

The DSC curve of Form CSIII is substantially as depicted in FIG. 2, which shows an endothermic peak at around 257° C. (onset temperature), corresponding to the melting endothermic peak.

Table 2

| Diffraction angle 2θ | d spacing | Intensity % |
|---|---|---|
| 6.43 | 13.75 | 26.90 |
| 10.14 | 8.73 | 14.16 |
| 11.33 | 7.81 | 85.41 |
| 12.66 | 6.99 | 36.22 |
| 14.57 | 6.08 | 3.38 |
| 15.39 | 5.76 | 2.83 |
| 16.31 | 5.43 | 9.38 |
| 16.48 | 5.38 | 10.12 |
| 18.35 | 4.83 | 4.87 |
| 18.53 | 4.79 | 8.34 |
| 19.29 | 4.60 | 21.72 |
| 19.91 | 4.46 | 7.20 |
| 20.21 | 4.39 | 3.58 |
| 20.59 | 4.31 | 23.95 |
| 21.55 | 4.12 | 3.07 |
| 21.94 | 4.05 | 3.45 |
| 22.77 | 3.91 | 16.96 |
| 23.21 | 3.83 | 100.00 |
| 25.02 | 3.56 | 6.76 |
| 25.93 | 3.44 | 8.87 |
| 26.34 | 3.38 | 2.49 |
| 26.70 | 3.34 | 3.61 |
| 27.08 | 3.29 | 4.07 |
| 27.53 | 3.24 | 3.11 |
| 27.79 | 3.21 | 9.66 |
| 28.43 | 3.14 | 2.58 |
| 28.63 | 3.12 | 3.18 |

### Example 2 Preparation of Form CSIII

2.3151 g of Compound I was weighed into a glass vial, and then 140 mL of N, N-dimethylacetamide was added to form a clear solution. The solution was filtered, and 5 mL of the filtrate was evaporated at 50° C. to obtain a solid. The crude solid was dried at 50° C. The obtained solid was confirmed to be Form CSIII of Compound I.

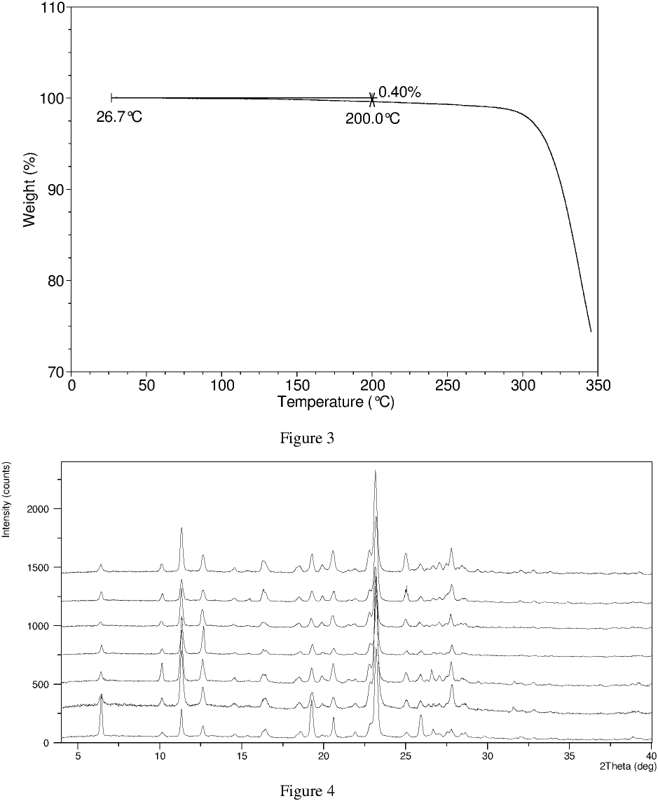

The TGA curve of Form CSIII is substantially as depicted in FIG. 3, which shows about 0.4% weight loss when heated to 200° C.

### Example 3 Preparation of Form CSIII

58.3 mg of Compound I was weighed into a glass vial, then 4 mL of N, N-dimethylacetamide was added to form a clear solution. The solution was filtered, and the filtrate was evaporated at 50° C. to obtain a solid. The crude solid was dried at 100° C. The obtained solid was confirmed to be Form CSIII.

The ¹H NMR data of Form CSIII are: 1H NMR (400 MHz, CDC13) δ 10.98 (s, 1H), 9.29 (s, 1H), 8.20 (s, 1H), 8.10 (s, 1H), 8.06 (s, 1H), 7.80 (dd, J = 7.9, 1.6 Hz, 1H), 7.51 (dd, J = 8.0, 1.5 Hz, 1H), 7.26 (t, J = 7.9 Hz, 1H), 4.00 (s, 3H), 3.81 (s, 3H), 1.79-1.72 (m, 1H), 1.16-1.06 (m, 2H), 0.93-0.86 (m, 2H)ₒ

### Example 4 Kinetic Solubility of Form CSIII

When solubility test is used to predict the in vivo performance of a drug, it is critical to simulate in vivo conditions as closely as possible. Simulated gastric fluid (SGF), Fed-state simulated intestinal fluid (FeSSIF) and Fasted-state simulated intestinal fluid (FaSSIF) can be used to simulate the conditions in vivo and predict the effects of eating, thus solubilities in these media are closer to those in vivo.

15-20 mg of Form CSIII of the present disclosure and 15-20 mg of Form A were suspended into 2.5 mL of SGF, 2.5 mL of FeSSIF, and 2.5 mL of FaSSIF to get suspensions. After equilibrated for 1 h and 4 h, concentrations of these solutions were measured by HPLC. The results are listed in Table 3.

Table 3

| Media | Form A | | Form CSIII | |
|---|---|---|---|---|
| | 1 hour | 4 hours | 1 hour | 4 hours |
| | Concentration mg/mL | Concentration mg/mL | Concentration mg/mL | Concentration mg/mL |
| SGF | 1.1141 | 0.6172 | 1.5824 | 1.2968 |
| FeSSIF | 0.0823 | 0.0688 | 0.1936 | 0.1793 |
| FaSSIF | 0.0135 | 0.0136 | 0.0490 | 0.0557 |

The results show that the solubility of Form CSIII in SGF, FeSSIF and FaSSIF are higher than those of Form A at 1 hour and 4 hours, indicating that Form CSIII has higher solubility.

### Example 5 Stability of Form CSIII

Approximately 5 mg of Form CSIII samples was stored under different conditions of 25° C./60%RH, 40° C./75%RH, and 60° C./75%RH. Crystalline form and chemical purity were checked by XRPD and HPLC, respectively. The results are shown in Table 4, and the XRPD overlay is shown in FIG. 4.

Table 4

| | | | |
|---|---|---|---|
| Initial form | Conditions | Time | Crystalline form |
| Form CSIII | Initial | | Form CSIII |
| | 25° C./60%RH (open) | 3 months | Form CSIII |
| | 25° C./60%RH (sealed) | 3 months | Form CSIII |
| | 40° C./75%RH (open) | 3 months | Form CSIII |
| | 40° C./75%RH (sealed) | 3 months | Form CSIII |
| | 60° C./75%RH (open) | one month | Form CSIII |
| | 60° C./75%RH (sealed) | one month | Form CSIII |

The results show that Form CSIII is stable for at least 3 months at 25° C./60% RH and 40° C./75% RH. Form CSIII is stable for at least one month at 60° C./75% RH with only 0.07% changes in chemical purity. Form CSIII has good stability under long-term, accelerated and stress conditions.

### Example 6 Physical Stability of Form CSIII Under Pressure

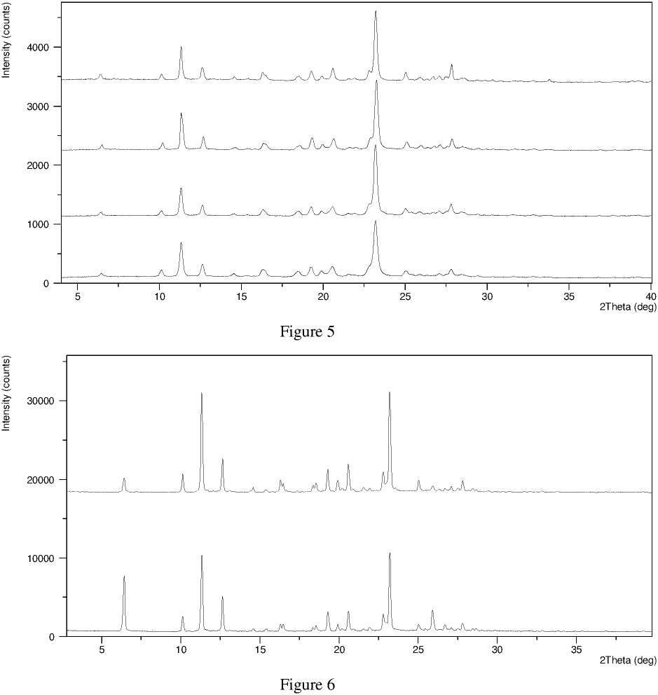

A certain amount of Form CSIII was compressed into tablets under 5 kN, 10 kN and 20 kN with suitable tableting die. Crystalline forms before and after tableting were checked by XRPD. The test results show that the crystalline state of Form CSIII does not change under different stress. The XRPD overlay is shown in FIG. 5.

### Example 7 Physical Stability of Form CSIII Upon Grinding

Form CSIII sample was grounded manually for 5 minutes in a mortar. Crystalline forms before and after grinding were checked by XRPD. The results show that the crystalline state of Form CSIII does not change before and after grinding. The XRPD overlay is shown in FIG. 6.

### Example 8 Physical Stability of Form CSIII Upon Humidity Change

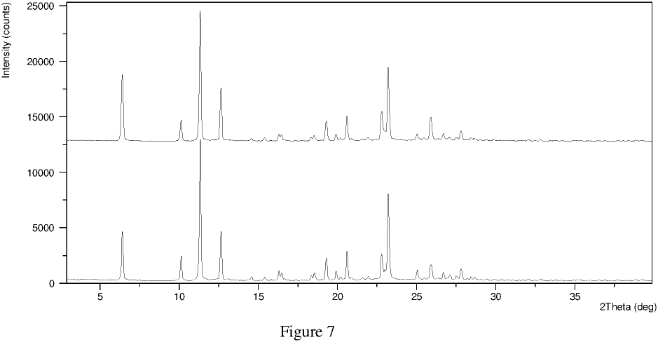

Dynamic vapor sorption (DVS) was applied to test the stability of Form CSIII. A certain amount of Form CSIII was tested in a cycle of 0%-95%-0%RH at 25° C. The XRPD overlay of Form CSIII before and after DVS test is shown in FIG. 7. The test results show that the crystalline state of Form CSIII does not change after DVS test, indicating that Form CSIII has superior property.

### Example 9 Density of Form CSIII

About 500 mg of powder were added into a 5-mL measuring cylinder and bulk volume was recorded. Then the powder was tapped for 1250 times by ZS-2E tap density tester to make it in the tightest state and the tapped volume was recorded. The bulk density and tapped density were calculated. Results of density of Form CSIII and Form A are shown in table 5.

Table 5

| Form | Bulk density (g/mL) | Tapped density (g/mL) |
|---|---|---|
| Form A | 0.3899 | 0.5199 |
| Form CSIII | 0.4276 | 0.5987 |

The results indicate that the density of Form CSIII is higher than that of Form A.

The examples described above are only for illustrating the technical concepts and features of the present disclosure and intended to make those skilled in the art being able to understand the present disclosure and thereby implement it and should not be concluded to limit the protective scope of this disclosure. Any equivalent variations or modifications according to the spirit of the present disclosure should be covered by the protective scope of the present disclosure.

1. A crystalline form CSIII of Compound I, wherein the X-ray powder diffraction pattern comprises characteristic peaks at 2theta values of 6.4°±0.2°, 11.3°±0.2° and 23.2°±0.2° using CuKα radiation

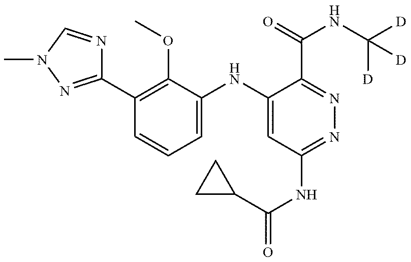 [SMILES: [2H]C([2H])([2H])NC(=O)c1nnc(NC(=O)C2CC2)cc1Nc1cccc(-c2ncn(C)n2)c1OC]

2. The crystalline form CSIII of Compound I according to claim 1, wherein the X-ray powder diffraction pattern comprises one or two or three characteristic peaks at 2theta values of 10.1°±0.2°, 12.7°±0.2° and 19.3°±0.2° using CuKα radiation.

3. The crystalline form CSIII of Compound I according to claim 1, wherein the X-ray powder diffraction pattern comprises one or two or three characteristic peaks at 2theta values of 20.6°±0.2°, 25.9°±0.2° and 27.8°±0.2° using CuKα radiation.

4. A crystalline form CSIII of Compound I, wherein, the X-ray powder diffraction pattern is substantially as depicted in FIG. 1.

5. A process for preparing crystalline form CSIII of Compound I according to claim 1, comprising: dissolving Compound I solid in an amide solvent, evaporating to obtain crystalline form CSIII.

6. A pharmaceutical composition, wherein said pharmaceutical composition comprises a therapeutically effective amount of crystalline form CSIII of Compound I according to claim 1 and pharmaceutically acceptable excipients.

7. (canceled)

8. (canceled)

9. A method of inhibiting TYK2 receptor, comprising administering to a subject in need thereof a therapeutically effective amount of crystalline form CSIII of Compound I according to claim 1.

10. A method for treating psoriasis, systemic lupus erythematosus, and Crohn’s disease, comprising administering to a subject in need thereof a therapeutically effective amount of crystalline form CSIII of Compound I according to claim 1.
